(19) United States
(12) Patent Application Publication
(10) Pub. No.: US 2011/0137213 A1
(43) Pub. Date: Jun. 9, 2011

(54) METHOD AND SYSTEM FOR THERAPEUTIC EXERGAMING

(75) Inventors: Brian CAULFIELD, Dublin (IE); Diarmaid FITZGERALD, Dublin (IE); Barry SMYTH, Bray (IE)

(73) Assignee: UNIVERSITY COLLEGE DUBLIN, NATIONAL UNIVERSITY OF IRELAND, DUBLIN, Dublin (IE)

(21) Appl. No.: 12/956,334

(22) Filed: Nov. 30, 2010

Related U.S. Application Data
(60) Provisional application No. 61/264,994, filed on Nov. 30, 2009.

(51) Int. Cl. A61B 5/11; H04W 88/02

(52) U.S. Cl. 600/595; 455/556.2

(57) ABSTRACT

A computer program product comprising a non-transitory computer readable medium for storing or recording instructions in machine readable form. The instructions, when executed in a motion sensor enabled smart phone, record target data associated with a response of the motion sensor to a desired sequence of movements of a user performing a therapeutic exercise while wearing the smart phone, map the target data to run-time target parameters of a software application, receive performance data associated with a response of the motion sensor to a subsequent sequence of movements of the user performing the therapeutic exercise while wearing the smart phone, map the performance data to the target parameters of the software application to determine the operation of the software application and provide information for display on a remote monitor visible to the user indicating the quality of the user's performance of the exercise.

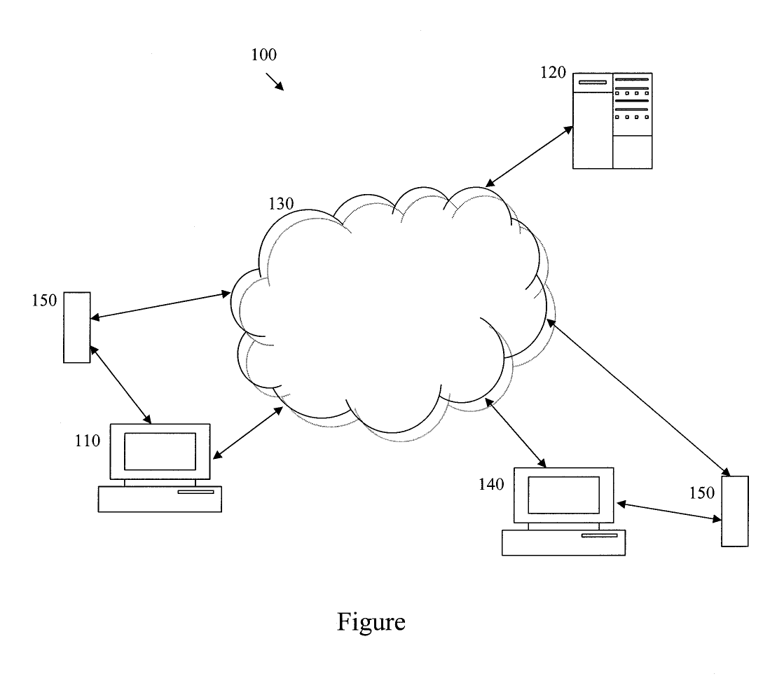

The present invention relates to a method and system for exergaming, and in particular, a motion sensor enabled smart phone comprising application software configured for therapeutic exergaming and a method operable with a motion sensor enabled smart phone for therapeutic exergaming.

### BACKGROUND OF THE INVENTION

Conventionally, physiotherapists, athletic trainers, strength and conditioning coaches and rehabilitation practitioners prescribe exercise therapy programmes to their clients as part of a treatment programme for many movement-impaired disorders and/or to enhance physical performance in sport.

However, poor adherence and inadequate exercise technique often result in poor outcomes for the patient, delaying their return to full physical function.

Thus, it is an object of the present invention is to provide a method and system for improving a patient's treatment programme.

### SUMMARY OF THE INVENTION

According to the present invention, there is provided application software for a motion sensor enabled smart phone, the application software which when executed being arranged to record target data associated with a response of the motion sensor to a desired sequence of movements of a user performing a therapeutic exercise while wearing the smart phone; map the target data to provide subsequent run-time target parameters of the application software; receive performance data associated with a response of the motion sensor to a subsequent sequence of movements of the user performing the therapeutic exercise while wearing the smart phone; map the performance data to the target parameters of the application to determine the operation of the application software; and provide information for display on a remote monitor visible to the user indicating the quality of the user's performance of the exercise.

Preferably, the application software comprises a module executable on the smart phone for recording the target data; and a separate module executable on the smart phone for receiving the performance data and providing the information for display.

Alternatively, or in addition, the application software comprises a module executable on the smart phone for streaming sensor data to a remote server or local computer with application software responsible for both recording target movement data and receiving performance data and providing information for display.

In a further aspect, there is provided a smart phone including a motion sensor and in which the above application software is installed.

According to a further aspect of the invention, there is provided a server in communication with a motion sensor enabled smart phone across a communications network, the server being arranged to: receive from application software running on the motion sensor enabled smart phone, target data associated with a response of the motion sensor to a desired sequence of movements of a user performing a therapeutic exercise while wearing the smart phone; map the target data to provide subsequent run-time target parameters of the application software; receive from the motion sensor enabled smart phone, performance data associated with a response of the motion sensor to a subsequent sequence of movements of the user performing the therapeutic exercise while wearing the smart phone; map the performance data to the target parameters of the application to determine the operation of the application software; and compare the performance data with the target data to determine the user's compliance with a therapeutic exercise regime.

A method of therapeutic exergaming operable with a motion sensor enabled smart phone, the method comprising devising a therapeutic exercise for a patient; attaching the motion sensor enabled smart phone to the patient; recording target data associated with a response of the motion sensor to a desired sequence of movements of the patient performing the therapeutic exercise while wearing the smart phone; mapping the target data to provide subsequent run-time target parameters of application software; receiving performance data associated with a response of the motion sensor to a subsequent sequence of movements of the patient performing a therapeutic exercise while wearing the smart phone; mapping the performance data to the target parameters of application software to determine the operation of the application software; and providing information for display on a remote monitor visible to the patient indicating the quality of the patient's performance of the exercise.

### BRIEF DESCRIPTION OF THE DRAWINGS

The FIGURE illustrates a communication system comprising first and second client nodes, a motion sensor enabled smart phone, and a remote server, each of the nodes, smart phone and remote server interconnected across a communications network in accordance with an embodiment of the present invention.

### DETAILED DESCRIPTION OF THE INVENTION

In recent years, the fields of exercise and computer games have been combined to create a new genre known as exergaming. Exergaming involves utilisation of motion sensing technologies to enable large body movements to be integrated into game-play.

A well-known example of an exergaming system is Nintendo's Wii console. The console comprises motion sensors arranged to determine movements of a player when playing a game thereby enabling players to participate in game-play in a natural manner, by simply performing the appropriate actions for the game, such as swinging a bat, bowling a ball or steering a car.

Therapeutic exergaming involves utilising computer games in conjunction with body-worn motion tracking sensors to monitor, teach and improve a patient's exercise or treatment programme. In particular, suites of computer games and/or haptic feedback modules are adapted by practitioners to provide a biofeedback of therapeutic exercise for aiding specific therapeutic exercise contexts and outcomes.

In accordance with one embodiment of the present invention, application software running on a motion sensor enabled “smart phone” is arranged to record, in the presence of a healthcare professional, game input data associated with a pattern of desired patient movements. In the context of the description, a “smart phone” is defined as a device that provides voice connectivity, data connectivity, and computerised application programs.

The input data is then translated or mapped to a gaming application running on the smart phone. Alternatively, the input data transmitted to a remote server on which a gaming application server runs, and is then translated or mapped before being transmitted back to the gaming application.

Once the mapping is complete, the patient can then access their personalized exercise programme plan from home or elsewhere by playing the gaming application. Game performance or feedback is then based on how well a patient moves in relation to the target pattern of body movement specific to the patient that has been designed and recorded by the heathcare professional.

Referring now to the FIGURE, there is illustrated a communication system 100 comprising a first client node 110 in communication with a remote server 120 across a communications network 130, and a second client node 140 in communication with the server 120 across the communications network 130. The network can comprise any suitable communications network including the Internet, 3G network, GSM, mesh network or otherwise. The first and second client nodes 110, and 140, are further arranged to communicate with a motion sensor enabled smart phone 150. It will be appreciated that the computers or nodes 110, 140 need not necessarily be general purpose computers and could for example comprise gaming consoles for example of the Sony Playstation, Nintendo Wii or Microsoft Xbox type.

In one embodiment of the present invention, the communications system of the FIGURE is utilised to provide a patient with a customised exercise programme and to enable a healthcare professional such as a physiotherapist or rehabilitation practitioner to monitor, teach and improve a patient's customised exercise programme/regime.

Firstly, the healthcare professional designs a specific exercise programme for a patient. The exercise program comprises a particular sequence of movements or game play, designed to guide the patient to reliably perform a particular sequence of body movements corresponding to a prescribed therapy.

In one embodiment, the practitioners avail of a user interface running on the first client node 110 or local computer, to set parameters for the exercise programme and these parameters are subsequently transmitted via the communications network 130 to the remote server 120 on which the gaming application is stored. The parameters may comprise for example, gaming environment, exercise choice, and difficulty level. However, it will be appreciated, that instead of using a local computer, the practitioner may avail of a user interface displayed on the smart phone 150 in order to set the customized patient parameters, particularly in the case that the gaming application is being stored on the smart phone.

Preferably, the practitioner employs conventional patient analysis tests to determine suitable parameters for a patient. For example, the star excursion balance test, (SEBT), as disclosed in Kinzey S J, Armstrong C W, “The reliability of the star excursion test in assessing dynamic balance”, J Orthop Soports Phys Ther. Vol. 27, No. 5, pp. 356-360, 1998, may be employed to assess balance control of a patient, and the Isokinetic dynamometry (Biodex System 3) may be employed to measure knee flexion and extension concentric strength and endurance of a patient.

The parameters allow for the practitioner to vary the difficulty of the game, thereby requiring the patient to perform their exercise routine more precisely. Preferably, the practitioner creates a number of different exercises with various durations and difficulty levels, each customized for a patient given their specific injuries and physical characteristics, to thereby produce a highly personalized exercise program for the patient.

In one embodiment, the interface also enables the practitioner to configure a response of the sensor to movement, for example, by selecting how each sensor axis relates to a particular game control. For example, a lunge exercise whereby a patient dips their body into the position of a lunge translates as a dip down of a flight path in a navigation computer game.

In order to create the desired game-play sequence, the practitioner instructs the patient to perform the desired exercise while wearing the motion sensor enabled smart phone 150 about a suitable location on their body. The application software running on the smart phone records the movements of the patient performing the exercise and translates or maps the recorded movements, or target data associated with the recorded movements, into a particular action in a game or virtual world so that when playing the game, the patient can relate their movements to their progression in the game.

Performing the desired exercise in the presence of the practitioner enables the practitioner to ensure that the patient is performing the exercises correctly and accordingly, that the target data being recorded represents data associated with a correctly performed exercise.

In one embodiment, the smart-phone 150 is applied to target areas of the patient's body by housing it in a pocket or a brace or strap (not shown) and securing it to the patient's body by means of Velcro straps. However it will be appreciated that any means of securing the smart-phone to the target area may be employed. Accurate positioning of the brace or strap is achieved by aligning key points indicated on the brace with anatomical landmarks.

In an alternative embodiment, the patient's recorded movements or target data associated with the recorded movements is transmitted to the remote server 120, and it is stored in conjunction with the parameters associated with the patient. In one embodiment, the recorded movements or input data is transmitted from the smart phone 150 to the remote server 120 by means of a 3G network. Alternatively, the input data is transmitted to the local computer 110, and is transmitted to the remote server 120 independently, or in conjunction with the patient's parameters. However, it will be appreciated that any suitable transmitting means may be employed to upload the input data and patient parameters to the remote server 120 on which the gaming application is stored.

Accordingly, in one embodiment, target data associated with the recorded movements is translated into a particular action of a gaming application stored on the smart phone 150. Optionally, the healthcare practitioner may set the parameters of the game on the smart phone. Preferably, these parameters are employed in conjunction with the target data to determine target parameters for the gaming application. The patient can access the customized gaming application on their smart phone, select an exercise to perform, and movements for which the sensor is configured.

In an alternative embodiment, where the gaming application is held at the remote server 120, the patient is preferably provided with login credentials to enable them to access the exercise programme stored on the remote server 120. To this end, a user interface running on a second client node 140 or local computer is employed to enable the patient to input their login credentials, which are sent across the network 130 to the remote server 120. Once verified, the patient can run the customized gaming application, select an exercise to perform, and movements for which the sensor is configured.

The gaming application is configured to drive display on a screen, or on a monitor associated with the local computer 140.

In one embodiment, the patient activates or instantiates the gaming application on the smart phone, attaches the smart phone 150 to the target area of their body, and performs the exercise whilst viewing the gaming application display on the screen or monitor. The application software running on the smart phone records performance data associated with a response of the motion sensor to a reproduced sequence of movements of the user and translates or maps the performance data to particular actions in the gaming application for display to the user. In one embodiment, the smart phone communicates with a monitor or screen, via a Bluetooth connection or any suitable means, to enable the game to be viewed by the patient while performing the exercise programme. The performance data is compared with the target data to determine the patient's compliance with the therapeutic exercise. The patient's progress is preferably transmitted to the remote server, from where it can be accessed by the healthcare professional.

In an alternative embodiment, where the gaming application is being held at the remote server 120, performance data corresponding to the patient's movements is recorded at the smart phone and transmitted from the smart phone 150 to the remote server 120 by means of the network 130. Alternatively, the performance data is transmitted to the local computer 140, and is subsequently transmitted to the remote server 120. However, it will be appreciated that any suitable transmitting means may be employed to upload the performance data to the remote server.

Game performance indicating the accuracy or precision with which the patient performs the relevant exercises is determined from a comparison of the performance data with the target pattern.

In one embodiment, during each game, the patient is presented with a sequence of checkpoints which tallies their progress and communicates it as a game score based on the accuracy with which they performed or reproduced the exercises of the programme. Each game is then saved for review by the practitioner. Preferably, as the patient progresses, progressively more challenging or alternative exercises may be devised which are more appropriate to late stage recovery. For example, in a car game, the car could be made to skid each time a patient over rotates their thigh inward or outward, in the coronal place, a common motor deficit seen in cases of musculoskeletal injuries.

In one embodiment, the results associated with a patient are compiled in a report or set of reports which can be arranged to provide an indication of a patient's progress in any suitable format, for example, day by day, week by week, or exercise by exercise. Preferably, the practitioner can review traces of individual exercises in order to make a more detailed examination of the progression of the patient and how and whether the prescribed exercise programme requires modification.

The invention is not limited to the embodiment(s) described herein but can be amended or modified without departing from the scope of the present invention.

1. A computer program product comprising a non-transitory computer readable medium for storing or recording instructions in machine readable form, which when executed in a motion sensor enabled smart phone, is arranged to:
record target data associated with a response of the motion sensor to a desired sequence of movements of a user performing a therapeutic exercise while wearing the smart phone;
derive run-time target parameters of a software application from the target data;
receive performance data associated with a response of the motion sensor to a subsequent sequence of movements of the user performing the therapeutic exercise while wearing the smart phone;
map the performance data to the target parameters of the software application to determine the operation of the software application; and
provide information for display on a remote monitor visible to the user indicating the quality of the user's performance of the exercise.

2. The computer program product of claim 1, comprising a module executable on the smart phone for recording the target data; and a separate module executable on the smart phone for receiving the performance data and providing the information for display.

3. The computer program product of claim 1, comprising a module executable on the smart phone for streaming sensor data to a remote server or local computer with application software responsible for both recording target movement data and receiving performance data and providing information for display.

4. The computer program product of claim 1, wherein said software application is a gaming application.

5. The computer program product of claim 1, further arranged to compare said performance data with said target data to determine a performance level associated with the user's ability to perform the exercise.

6. The computer program product of claim 1, further arranged to record input data for indicating software application preferences, wherein said input data comprises at least one of gaming environment, exercise choice, duration, and difficulty level.

7. The computer program product of claim 1, wherein the target data is recorded while the user is subjected to a patient analysis test by a practitioner of the therapeutic exercise.

8. The computer program product of claim 7, wherein the patient analysis test is a star excursion balance test.

9. The computer program product of claim 1, further arranged to receive a configuration input, wherein the configuration input dictates a response of the sensor to movement.

10. The computer program product of claim 9, wherein the configuration input relates to a particular game control of the software application.

11. The computer program product of claim 1, wherein the mapping of the target data to run-time target parameters of the software application corresponds with a mapping of the recorded movements of the user into a particular action in the application software.

12. A smart phone including a motion sensor and a computer program product according to claim 1.

13. The smart phone of claim 12 further comprising a fastener to enable the smart phone to be attached to the body of the user.

14. A server in communication with a motion sensor enabled smart phone across a communications network, the server being arranged to:
receive from a software application running on the motion sensor enabled smart phone, target data associated with a response of the motion sensor to a desired sequence of movements of a user performing a therapeutic exercise while wearing the smart phone;
derive run-time target parameters of a software application from the target data;
receive from the motion sensor enabled smart phone, performance data associated with a response of the motion sensor to a subsequent sequence of movements of the user performing the therapeutic exercise while wearing the smart phone;
map the performance data to the target parameters of the software application to determine the operation of the software application; and
compare the performance data with the target data to determine the user's compliance with a therapeutic exercise regime.

15. A method of therapeutic exergaming operable with a motion sensor enabled smart phone, the method comprising:
devising a therapeutic exercise for a patient;
attaching the motion sensor enabled smart phone to the patient;
recording target data associated with a response of the motion sensor to a desired sequence of movements of the patient performing the therapeutic exercise while wearing the smart phone;
deriving run-time target parameters of a software application from the target data;
receiving performance data associated with a response of the motion sensor to a subsequent sequence of movements of the patient performing a therapeutic exercise while wearing the smart phone;
mapping the performance data to the target parameters of the software application to determine the operation of the software application; and
providing information for display on a remote monitor visible to the patient indicating the quality of the patient's performance of the exercise.
